(19) United States
(12) Patent Application Publication
(10) Pub. No.: US 2022/0088017 A1
(43) Pub. Date: Mar. 24, 2022

(54) COMPOSITIONS AND METHODS FOR TREATING VIRAL INFECTIONS

(71) Applicants: Harun Kimani, Nairobi (KE); Walter Drane, Dripping Springs, TX (US)

(72) Inventor: Harun Kimani, Nairobi (KE)

(73) Assignees: Harun Kimani, Nairobi (KE); Walter Drane, Dripping Springs, TX (US)

(21) Appl. No.: 17/476,560

(22) Filed: Sep. 16, 2021

Related U.S. Application Data
(60) Provisional application No. 63/081,384, filed on Sep. 22, 2020.

(51) Int. Cl. A61K 31/505; A61K 47/44; A61P 9/00

(52) U.S. Cl. CPC A61K 31/505; A61P 9/00; A61K 47/44

(57) ABSTRACT

Preferred embodiments described herein relate to methods for preventing, treating, or reducing symptoms of viral infections in patients by administering a composition comprising one or more statins. The viral infections may be coronavirus infections and the administration of the statins may reduce vascular inflammation in the patient, leading to prevention of heart failure and related complications.

This application claims priority to U.S. Provisional Patent Application Ser. No. 63/081,384, entitled “Compositions and Methods for Treating Viral Infections,” filed Sep. 22, 2020, the entire contents of which are incorporated by reference herein.

### BACKGROUND

This disclosure pertains to compositions and methods for treatment of and prevention of complications arising from viral infections, particularly coronavirus infections.

COVID-19 is a disease caused by a new virus whose presentation has been noted to vary and thus the cause of pathology is not well described. The new corona virus has disrupted life as we knew it due to fear of infection and mortality as a result of the disease. The resultant widespread mortality has led it to be labeled a pandemic for affecting all continents in the world. Mortality has been varied and in some cases has defied projections by epidemiologic models. The cause of mortality and the ability to prevent it is of great interest to physicians and epidemiologists. Due to unknown reasons, the mortality has not followed projected mathematical models in some populations, thus raising the question of probable mitigation measures. As the world awaits definitive prevention of the disease using vaccination, environmental methods of social distancing have been recommended with limited success as shown by continued spread of disease in varied populations. Epidemiologic models that target host, agent and environment can be helpful in explaining interventions required to reduce mortality from the new virus. The coronavirus is both highly infectious and pathogenic and prevention of mortality under secondary prevention is highly desirable. Economic burden of coronavirus has led to unemployment and food insecurity as well as reduced uptake of health services.

### SUMMARY

The present disclosure relates generally to compositions and methods for treating viral infections, particularly infections with coronavirus.

Preferred embodiments described herein relate to the use of statins in treating symptoms and reducing complications arising from coronavirus infection. Statins include atorvastin, fluvastatin, lovastatin, pitavastatin, pravastatin, rosuvastatin, and simvastatin. Statins are HMG-CoA reductase inhibitors and are a class of drugs typically used to reduce illness and mortality in those who are at high risk of cardiovascular disease. In preferred embodiments described herein, statins are utilized to prevent heart disease in coronavirus patients with pre-existing diseases. The statin drugs can be from natural or synthetic sources and are shown to prevent the complications due to COVID.

The outcome of infection with coronavirus, particularly COVID-19, appears to be determined by host characteristics and response to the infection. Young people tend to have mild uncomplicated disease while older people and especially older men have complications as a result of the disease. Statins have been shown to prevent blood from sticking to blood vessels of coronavirus patients allowing time for the inflammation of the vessels to subside and heal. This shows that the complications that result in death can be prevented using statins. Host characteristics especially age and gender affect presentation of the viral infection and affect the outcome and complications thereof. Treatment with statins produces a positive outcome for the COVID-19 with pre-existing conditions. Vascular inflammation is later sequelae of coronavirus infection and more pronounced for those with co-existing conditions and prevented with the use of statins. Comorbidities are associated with the severity of COVID-19 progression due to blood sticking to blood vessel walls, which statins stop. It is thought that this blood sticking to blood vessel walls is the cause of right heart failure and sudden death which has been observed in middle aged men and elderly patients. Prevention of the complication by taking statins, either natural or synthetic, reduces mortality due to sudden circulation failure. The prevention of the effect of the coronavirus on circulation and the heart through the use of statins is key to reducing widespread death caused by the virus. This should target the vascular wall and the blood coagulation pathways in severe cases.

### BRIEF DESCRIPTION OF THE DRAWINGS

FIG. 1 shows a schematic of an exemplary pipeline framework for use in preferred embodiments of the methods and systems described herein.

### DETAILED DESCRIPTION OF PREFERRED EMBODIMENTS

The present disclosure relates to compositions and methods for treatment of symptoms and prevention of complications relating to viral infections, particularly infection with coronavirus.

Preferred embodiments described herein relate to the use of statins to prevent, treat, and reduce complications caused by coronavirus infections in both men and women. Statins are known to lower cholesterol, reduce coronary heart disease, and generally reduce cardiovascular disease in men and women, through the action of inhibiting HMG-CoA reductase. In this disclosure, statins are also shown to reduce stasis of blood caused by inflamed blood vessels, stop right heart failure, and prevent heart failure in coronavirus patients.

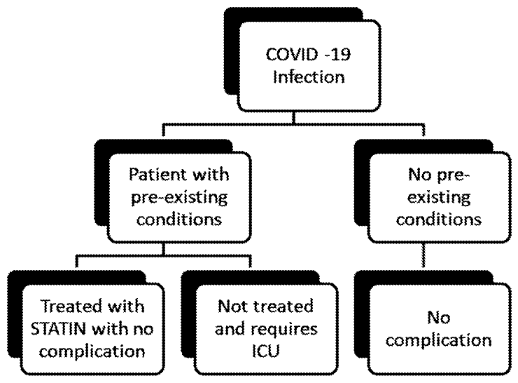

FIG. 1 shows a flow chart for prevention of vascular inflammation in patients infected with COVID-19. Patients with no pre-existing conditions tend to have no complications. Of patients with pre-existing conditions, those treated with statins also tend to have no complications. Patients with pre-existing conditions who were not treated tend to require ICU.

Men of age above 45 years infected with coronavirus tend to develop vascular inflammation resulting in circulatory blockage, right heart failure and mortality due to cardiac arrest which is prevented by the administration of statins as described herein. The same pathophysiology is believed to cause death in women who are above 60 years and patients with pre-existing conditions which make them prone to atherosclerosis. The statin-based medication described herein prevents death from circulatory blockage due to the activities of the statin.

Preferred embodiments disclosed herein include a method for preventing, treating, or reducing complications caused by coronavirus comprising the step of administering a statin to a patient infected with coronavirus. In preferred embodiments, the patient suffers from pre-existing conditions such as diabetes, obesity, asthma, hypertension, coronary heart disease, and other cardio-vascular diseases. The statin is preferably atorvastin, fluvastatin, lovastatin, pitavastatin, pravastatin, rosuvastatin, and simvastatin. In additional preferred embodiments, the statin is rosuvastatin. The statin may be administered at about 5 to about 25 milligrams daily, or at about 10 to about 20 milligrams daily, or at about 10 milligrams daily, for a period of time between one and three weeks, preferably about two weeks. The dosage can be increased depending on the level of atherosclerosis risk. In further preferred embodiments, the statin is administered in conjunction with a OMEGA 3 based vitamin oil, which is preferably administered as one capsule daily for a period of time between one and three weeks, preferably about two weeks.

In another aspect of the present invention there is provided a pharmaceutical composition including a therapeutically effective amount of a statin and a pharmaceutically acceptable excipient, adjuvant, carrier, buffer or stabilizer. A “therapeutically effective amount” is to be understood as an amount of an exemplary statin that is sufficient to show a positive biological effect on a coronavirus related symptom or condition being treated. The actual amount, rate and time-course of administration will depend on the nature and severity of the disease being treated. Prescription of treatment is within the responsibility of general practitioners and other medical doctors. The pharmaceutically acceptable excipient, adjuvant, carrier, buffer or stabilizer should be non-toxic and should not interfere with the efficacy of the active ingredient. The precise nature of the carrier or other material will depend on the route of administration, which may be oral, or by injection, such as cutaneous, subcutaneous, or intravenous injection, or by dry powder inhaler.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such as gelatin. For intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as sodium chloride solution, Ringer's solution, or lactated Ringer's solution. Preservatives, stabilizers, buffers, antioxidants and/or other additives may be included as required.

In another aspect, there is provided the use in the manufacture of a medicament of a therapeutically effective amount of a statin as defined above for administration to a subject.

The term “pharmacologically acceptable salt” used throughout the specification is to be taken as meaning any acid or base derived salt formed from hydrochloric, sulfuric, phosphoric, acetic, citric, oxalic, malonic, salicylic, malic, fumaric, succinic, ascorbic, maleic, methanesulfonic, isotonic acids and the like, and potassium carbonate, sodium or potassium hydroxide, ammonia, triethylamine, triethanolamine and the like.

The term “prodrug” means a pharmacological substance that is administered in an inactive, or significantly less active, form. Once administered, the prodrug is metabolised in vivo into an active metabolite.

The term “therapeutically effective amount” means a nontoxic but sufficient amount of the drug to provide the desired therapeutic effect. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. Furthermore, the effective amount is the concentration that is within a range sufficient to permit ready application of the formulation so as to deliver an amount of the drug that is within a therapeutically effective range.

Further aspects of the present invention will become apparent from the following description given by way of example only.

### Example 1. Observational

This example is an observational case study discussing clinical presentation of a patient as the index case and her contacts. The patient is a 35 years old female who works in a hospital as a general health care worker commonly referred as nurse-aide in Kenya. She presented with chest pain, fever and cough and was treated as an outpatient for a suspected chest infection. She did not improve and was admitted after developing difficulty in breathing 2 days later. This time a diagnosis of a suspected COVID-19 case was made and the patient was taken to the red zone awaiting further investigations. She received a chest x-ray, CT-scan and PCR test for coronavirus—which was positive. Patient remained dyspoenic and was put on oxygen and steroids. The chest x-ray showed massive pleural effusion on the right side of the chest. It was suggested that a chest tube be inserted for under-water seal drainage but this was not immediately done due to non-availability of the tube in hospital stock. Diagnostic pleural tap was done and noted that it was bloody and hemorrhagic in nature and which was taken for culture, sensitivity and cytology. CT-scan radiology specialist thought that the bloody effusion was due to a tumor but debris deposition was concluded to be the most likely cause. Staining for TB was negative and culture and sensitivity did not yield any bacteria. The patient was prescribed antibiotics for 6 weeks with serial X-rays to be done to follow effusion progression. Under-water seal drainage was not done even when the drainage kit became available as the patient seemed to stabilize on conservative management. Patient was released to homecare management where she is reported to be improving with occasional dyspnoea on exertion.

The presentation of the index case enabled the medical team to describe blistering of respiratory mucosa as the key cause of severe lung pathology. Until now it was thought that lung injury was a result of viral infection. The blisters that caused hemorrhagic pleural effusion are caused by lack of integrity of the junction between epithelial cells and the smooth muscles of the respiratory system. Upon squamation, there was resultant pleural effusion that appeared bloody. Debris deposited in the pleural cavity was mistaken for a tumor process. The viral infection by the coronavirus appears to be causing a chicken pox like blistering to the respiratory epithelium and not to the skin.

The other study subjects were the workmate contacts of the index case and their family members who were subsequently infected with coronavirus.

### Example 2. Prevention of Complications with Older Contacts

Older contacts of the index case from Example 1 who developed coronavirus showed signs of developing other complications despite acute phase of the disease being suppressed.

An older male of 50 years developed signs and symptoms of vascular inflammation and right heart failure. This included occasional headaches, flashes in vision, general fatigue, irritability, muscle pains, epigastric pain, general abdominal distention, inability to eat and circulatory congestion, salty or blood taste in sputum. The older male of 50 years was treated with 20 mg rosuvastatin daily for two weeks, concomitantly with an OMEGA 3 based vitamin oil also administered daily for two weeks which prevented this patient from going into right heart failure as the medical team diagnosed impending heart failure. The impending right heart failure was thought to be secondary to myocarditis, circulatory congestion due to poor blood flow as a result of vascular inflammation and sticky walls of blood vessels. The male contact was within 6 hours having intermittent chest pains, sweating, and dizziness. Cardiac arrest was thought to be a possible poor outcome leading to mortality. The medical team discussed interventions necessary with statins, with the intention of reducing blood sticking to blood vessels. The administered statin restored circulation and reversed impending right heart failure. The patient commenced treatment immediately with good results in 3 days' time. The abdominal swelling which was A sign of right heart failure subsided, abdominal pain reduced and the patient was able to eat with circulatory congestion eased. The administered statin showed good results in stopping right heart failure and preventing complications that lead to death in COVID-19 patients.

As discussed above, the key causes of pathology due to the viral infection were identified as respiration system blisters and vascular inflammation whose complications were prevented by commonly used anti-cholesterol drugs referred to as statins, by preventing development of acute atherosclerosis. The interventions were key to mitigate against the coronavirus complications, thus stopping mortality due to COVID.

Although the vast majority of contacts of confirmed COVID-19 cases do not suffer symptomatic or severe disease, a local medicinal concoction of honey, ginger, lemon and garlic is also expected to have effects on COVID-19 pathology. Ginger is thought to have generalizing anti-inflammatory effects. Two older females aged between 45 to 60 years who were contacts of the patient from Example 1 had more prolonged signs of viral infection for up to a week which included headaches, general malaise, occasional cough and insomnia. These were alleviated by administration of an antiviral concoction which consisted of freshly ground ginger, garlic, lemon and honey for a period of a week. Ginger has a characteristic flavour due to volatile oils of zingerone, shogaols and gingerols as well as enzymes zingiban and cysteine protease. Female gender with viral suppressant mixture seemed to be protective against severe disease. No side-effects were reported by the contacts. It was noted that blisters had started developing in pharyngeal mucosa but dried up on taking the natural anti-respiratory virus mixture.

| | USE OF STATIN | USE OF STATIN IN PREFERRED |
|---|---|---|
| Condition | NOW | EMBODIMENTS HEREIN |
| Blood | Lowering cholesterol | Reduce Stasis of blood |
| vessels | | |
| Heart | Reduce coronary heart | Stop right heart failure |
| Disease | disease | |
| Prevention | Primary prevention | Secondary prevention of heart failure |
| Gender | Lower cardiovascular | Reduce COVID complications in men |
| | diseases in men and | and women |
| | women | |
| Action | Inhibit HMG-CoA | Reduce stasis caused by inflamed |
| | reductase | blood vessels as a result of COVID |

### REFERENCES

The following publications are incorporated by reference herein.
Cascella M, Rajnik M, Cuomo A, Dulebohn S C & Di Napoli R. (2020) Features, evaluation and treatment coronavirus (COVID-19). Statpearls [internet]. StatPearls Publishing.
Chibwana M G, Jere K C, Mandolo J, Katunga-Phiri V, Tembo D, Mitole N, Musasa S, Sichone S, Lakudzala A & Sibale L. (2020) High SARS-CoV-2 seroprevalence in Health Care Workers but relatively low numbers of deaths in urban Malawi. medRxiv.
Linda N. (2020) The pandemic appears to have spared Africa. Science.
MOH. (2020) Interim Guidelines on Management of COVID-19 in Kenya. Kenya: Ministry of Health, Kenya.
Nicola M, Alsafi Z, Sohrabi C, Kerwan A, Al-Jabir A, losifidis C, Agha M & Agha R. (2020) The socio-economic implications of the coronavirus pandemic (COVID-19): A review. International journal of surgery (London, England) 78: 185.
Sanyaolu A, Okorie C, Marinkovic A, Patidar R, Younis K, Desai P, Hosein Z, Padda I, Mangat J & Altaf M. (2020) Comorbidity and its Impact on Patients with COVID-19. Sn Comprehensive Clinical Medicine: 1-8.
Tabary M, Khanmohammadi S, Araghi F, Dadkhahfar S & Tavangar S M. (2020) Pathologic features of COVID-19: A concise review. Pathol Res Pract 216: 153097.
Uyoga S, Adetifa I M, Karanja H K, Nyagwange J, Tuju J, Wanjiku P, Aman R, Mwangangi M, Amoth P & Kasera K. (2020) Seroprevalence of anti-SARS-CoV-2 IgG antibodies in Kenyan blood donors. medRxiv.
WHO. (2020a) Coronavirus disease (COVID-19) advice for the public. Available at: https://www.who.int/emergencies/diseases/novel-coronavirus-2019/advice-for-public.
WHO. (2020b) Kenya. Available at: https://covid19.who.int/region/afro/country/ke.
WHO. (2020c) WHO Coronavirus Disease (COVID-19) Dashboard. Available at: https://covid19.who.int/?gclid=CjwKCAjwkJj6BRA-EiwAOZVPVoWUarKOBPcX_tqSAx3eNNyxw1gBMAKr7qACU_qaDQ7pMxJfq74cFR oCOZEQAvD_BwE.
Xu X, Yu C, Qu J, Zhang L, Jiang S, Huang D, Chen B, Zhang Z, Guan W, Ling Z, Jiang R, Hu T, Ding Y, Lin L, Gan Q, Luo L, Tang X & Liu J. (2020) Imaging and clinical features of patients with 2019 novel coronavirus SARS-CoV-2. European journal of nuclear medicine and molecular imaging 47: 1275-1280.

What is claimed is:

1. A method for preventing, treating or reducing symptoms of a coronavirus infection in a patient, comprising:
administering a composition comprising one or more statins to the patient, whereby the composition reduces vascular inflammation in the patient.

2. The method of claim 1, wherein the one or more statins is atorvastin, fluvastatin, lovastatin, pitavastatin, pravastatin, rosuvastatin, simvastatin, or combinations thereof.

3. The method of claim 1, wherein the composition comprises from about 5 to about 25 milligrams of the one or more statins.

4. The method of claim 1, wherein the composition further comprises an OMEGA 3 based vitamin oil.

5. The method of claim 1, wherein the patient suffers from pre-existing conditions.

6. The method of claim 1, further comprising the step of observing a prevention or reduction in symptoms of the coronavirus infection in the patient.

7. The method of claim 6, wherein the symptoms comprise heart failure.

8. The method of claim 1, wherein the step of administering the composition occurs once daily for about one to three weeks.

9. The method of claim 1, wherein the one or more statins is rosuvastatin, and wherein the composition comprises from about 10 to about 20 milligrams of rosuvastatin.
